(19) United States
(12) Patent Application Publication
(10) Pub. No.: US 2024/0307608 A1
(43) Pub. Date: Sep. 19, 2024

(54) HYDRODYNAMIC FLUID THERAPY SYSTEMS AND METHODS FOR BURNS

(71) Applicant: Treysta Medical Technologies LLC, Gainesville, FL (US)

(72) Inventors: Nathan ZARNSTORFF, Gainesville, FL (US); Marcelo BUZZI, Newberry, FL (US)

(21) Appl. No.: 18/608,139

(22) Filed: Mar. 18, 2024

Related U.S. Application Data
(60) Provisional application No. 63/490,623, filed on Mar. 16, 2023.

(51) Int. Cl. A61M 3/02; A61B 17/3203

(52) U.S. Cl. CPC A61M 3/0202; A61B 17/3203; A61M 3/0229; A61M 2205/3334; A61M 2205/3344; A61M 2205/3368

(57) ABSTRACT

The subject invention pertains to a novel three-part system that delivers a series of medicated wound treatment solutions that provide precise biochemical conditions optimized for high-quality burn-wound healing. The system can include several key components, including a Hydrodynamic Dressing, one or more treatment solutions comprising novel and proprietary Therapeutic Fluids, and an Automated Fluid Delivery System that delivers the treatment solutions at an optimal flow rate within the Hydrodynamic Dressing. The Hydrodynamic Dressing can include a wound enclosure that isolates the wound from the nosocomial environment, inhibits infection, reduces the need for pain and trauma to the wound site during a dressing change, facilitates wound observation, and facilitates hydrodynamic debridement. The wound can be treated with each treatment solution in sequence, at specified flow rates, pressures, and temperatures by an automated conditioning and pumping unit for superior burn wound healing and overall better patient outcomes.

### CROSS-REFERENCE TO RELATED APPLICATION

The present application claims the benefit of U.S. Provisional Application Ser. No. 63/490,623, filed Mar. 16, 2023, which is hereby incorporated by reference herein in its entirety, including any figures, tables, nucleic acid sequences, amino acid sequences, or drawings.

### BACKGROUND OF THE INVENTION

Under the current standard of care, second and third-degree burns are debrided by surgical, chemical, mechanical, or autolytic methods and dressed and re-dressed throughout the healing process with an array of traditional medicated dressings. Although biological dressings have made great strides in stimulating tissue growth in burn wounds, these treatments remain painful, harmful to healing tissue, and result in both high cost and unfavorable aesthetic outcomes.

### BRIEF SUMMARY OF THE INVENTION

Embodiments of the subject invention provide a novel three-part system that delivers a series of medicated wound treatment solutions that provide precise biochemical conditions optimized for high-quality burn-wound healing. In certain embodiments, the device includes several key components, including but not limited to: a Hydrodynamic Dressing Pouch, one or more treatment solutions comprising novel and proprietary Therapeutic Fluids, and an Automated Fluid Delivery System that delivers the treatment solutions at the optimal flow rate within the Hydrodynamic Dressing Pouch. The Hydrodynamic Dressing Pouch comprises a wound enclosure that isolates the wound from the nosocomial environment, inhibits infection, reduces the need for pain and trauma to the wound site during a dressing change, facilitates wound observation, and facilitates hydrodynamic debridement. The wound can be treated with each treatment solution in sequence, at specified flow rates, pressures, and temperatures by an automated conditioning and pumping unit for superior burn wound healing and overall better patient outcomes.

Embodiments comprising the Hydrodynamic Dressing Pouch provide a revolutionary wound enclosure that allows for gentle wound irrigation in microbial isolation from the nosocomial environment. A distributed array of nozzles within each pouch can deliver treatment fluids at measured rates to facilitate the removal of toxins and necrotic byproducts from the wound site. Fluids that pass across the wound bed can be routed to a waste container.

Because direct wound-dressing contact is eliminated by certain embodiments, painful dressing change events and the associated trauma to the wound can be reduced, eliminated, avoided, or delayed in the weeks or months following a severe burn. Certain embodiments also provide a transparent design the also allows health care providers to observe the wound while using an integrated secondary directable fluid pressure and flow nozzle to gently hydrodynamically remove necrotic tissues without the pain or microbial risk associated with the removal of the dressing.

According to certain embodiments, within the hydrodynamic pouch a series of topical irrigation fluids (e.g., fluids comprising Treatment Solutions) can suffuse the pouch to accomplish various therapeutic actions. These fluids and the provided Treatment Solutions can provide intense hydration conducive to wound healing, restore normal osmotic pressure in the burned tissue, chemically and hydrodynamically micro-debride and flush away necrotic byproducts and oxidizing compounds, remove bacterial biofilm, maintain a hygienic wound environment, and inhibit various undesirable effects including but not limited to inflammation, edema, and excessive fibrous tissue proliferation.

Embodiments provide an Automated Fluid Delivery System wherein the delivery of Therapeutic Fluids can be managed by an automatic, programmable liquid flow control unit designed to deliver treatment solutions according to a specific regimen, with optimal flow rates, pressures, and temperatures. This combination of conditioning and flow control allows the overall system to generate an optimal wound environment for high-quality healing outcomes. In addition, a high-speed secondary pump allows for practitioner-directed, gentle, hydrodynamic debridement within the pouch itself.

### BRIEF DESCRIPTION OF THE DRAWINGS

FIG. 1 Schematically illustrates a Hydrodynamic Dressing Pouch adapted to treat a burn on or near a hand according to an embodiment of the subject invention.

FIG. 2 Schematically illustrates an Automated Fluid Delivery System connected to a Hydrodynamic Dressing Pouch adapted to treat a burn on the dorsal area of a patient according to an embodiment of the subject invention.

FIGS. 3A-3F Schematically illustrate respective Hydrodynamic Dressing designs adapted to treat burns in specific areas of patient anatomy according to certain embodiments of the subject invention.

FIG. 4 Schematically illustrates an Automated Fluid Delivery System according to an embodiment of the subject invention.

FIG. 5 Schematically illustrates a functional taxonomy organizing respective Hydrodynamic Dressing designs adapted to treat burns in specific areas of patient anatomy according to certain embodiments of the subject invention.

### DETAILED DISCLOSURE OF THE INVENTION

The current standard of care dictates that dressing changes should be frequent enough to control exudate but not so frequent that they interfere with wound re-epithelialization. Changing a traditional dressing causes trauma to the epithelializing tissue and often causes significant patient pain. Dressing change frequency ranges from twice daily to weekly, depending upon the amount of exudate, the presence of infection, and the choice of dressing material. Embodiments of the provided hydrodynamic dressing can replace traditional dressings, allowing the entire burn region to be continuously submerged in therapeutic fluids. As a result, dressing adhesion, trauma to the wound site, and the pain associated with dressing change can be inhibited, reduced, or in some cases, eliminated.

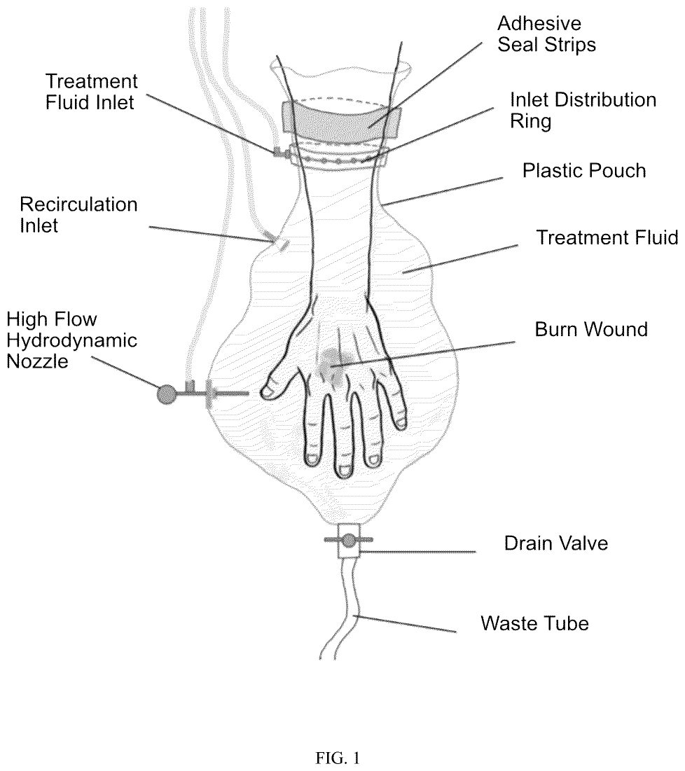

In certain embodiments, the provided Hydrodynamic Dressing Pouch isolates the area around the wound from the surrounding environment and can be transparent, waterproof, and sterile. The provided pouch can be anatomically shaped for enhanced adhesion to the skin. Embodiments provide a flexible and malleable biocompatible polymer pouch tailored to the peri-wound area and configured to hold an adequate volume of therapeutic fluid while reducing, eliminating, or effectively eliminating leakage or unwanted entry of air and contaminants into the fluid phase around the wound area. Embodiments provide a pouch that is transparent, pliable, and flexible to enable a burn care practitioner to monitor the clinical development of the wound continuously, periodically, or as needed, and to perform manual debridement to assist in the detachment of devitalized tissues. The application of malleable, flexible, and transparent materials such as medical-grade PVC in manufacturing the pouch can be advantageous. Due to the flexible and impermeable material, the hydrodynamic dressing pouch can be molded into different shapes and sizes to accommodate the burned patient's limbs and body parts. A directional jet nozzle can be attached within the pouch wall and connected to a high-performance peristaltic pumping system to drive fluids internal to the fluid pouch, providing the burn care expert access to a hydrodynamic debridement instrument in a fully sterile fluidic phase. In certain embodiments, a Hydrodynamic Dressing Pouch, specific for hand burns, can be in the form of a bag with a relatively narrow mouth where the hands are dressed (e.g., FIG. 1). In this opening, certain embodiments provide silicone or polyurethane foam tape for scaling the pouch using a specific hydrogel glue for holding the fluid inside the bag. The inlet for the treatment fluid can be distributed in a plastic ring with several holes that uniformly irrigate all around the arm. This same (or a similar) configuration of the Hydrodynamic Dressing Pouch for Hands can also be adapted for treatment of burns on lower extremities such as the feet and legs, adjusting the shape and sizing of the pouch to accommodate specific anatomical features.

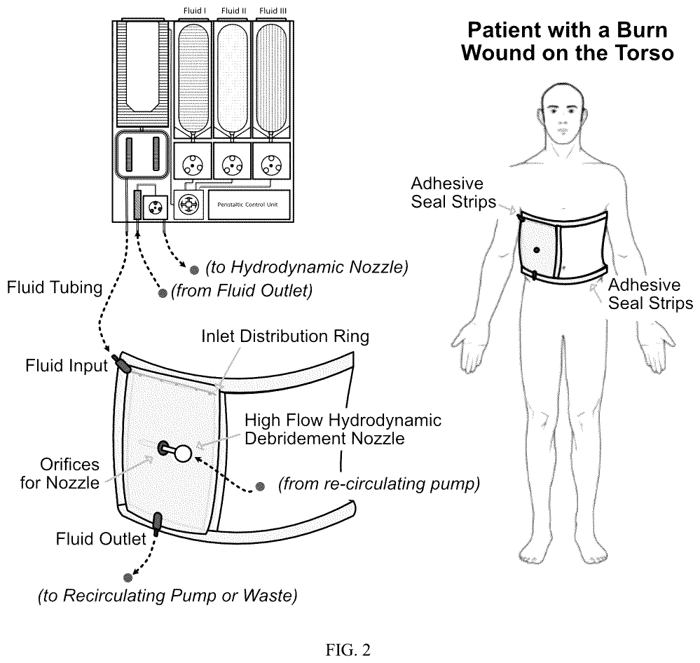

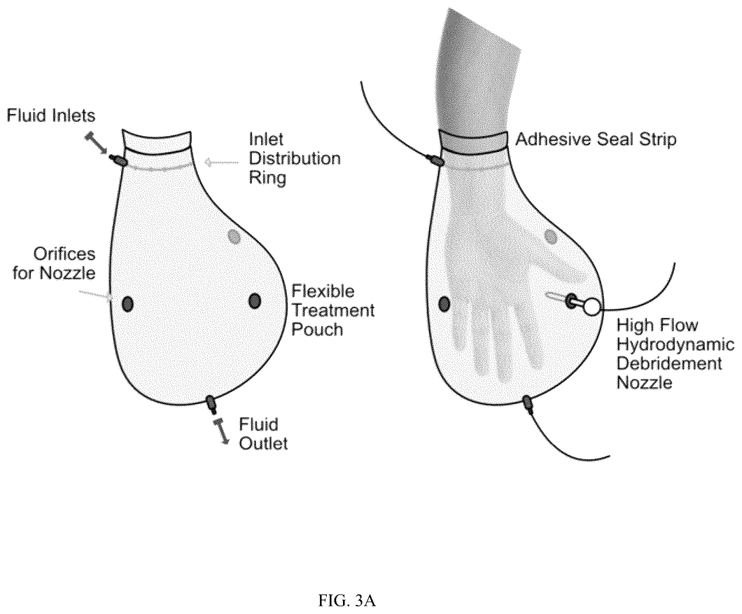

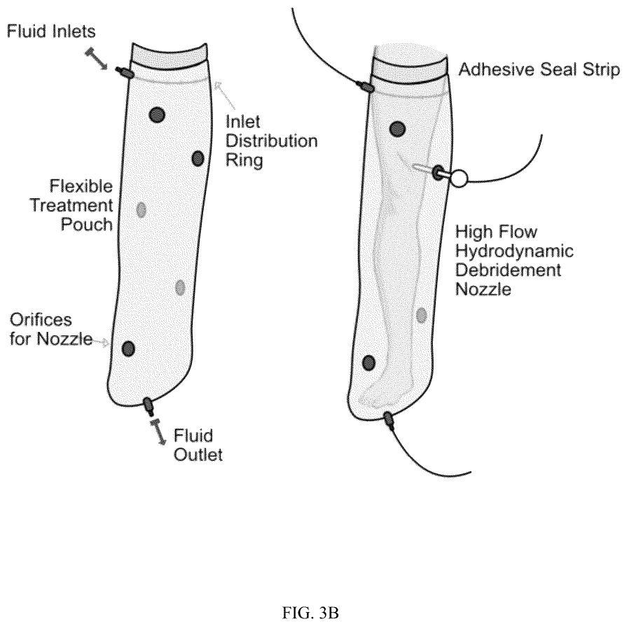

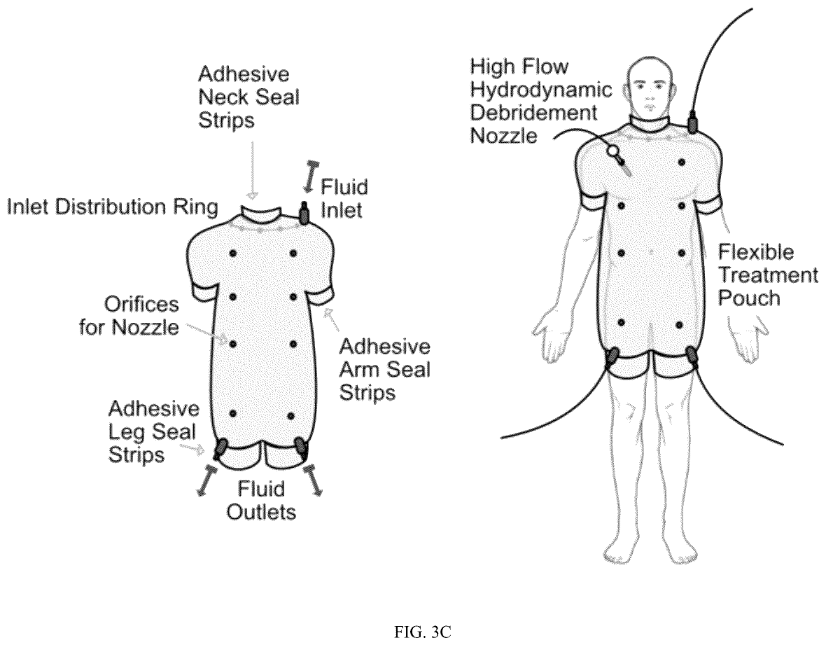

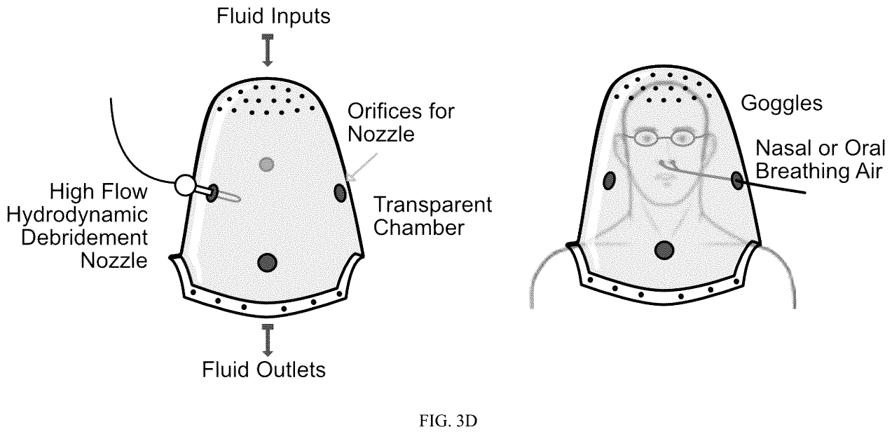

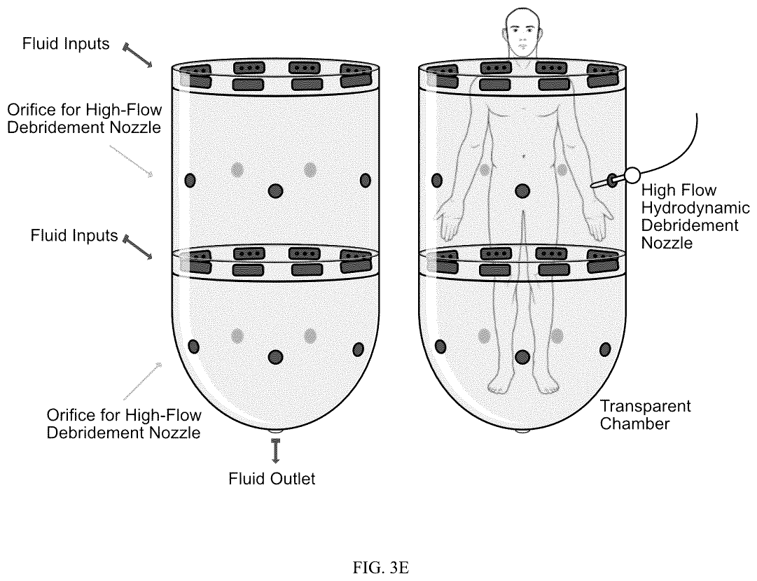

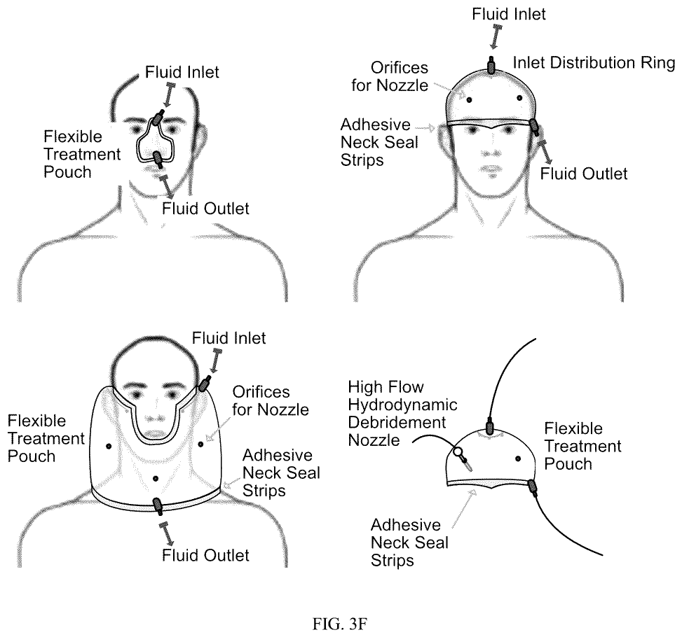

In other areas of the body (e.g., in the thorax, abdomen, and back), the Hydrodynamic Dressing Pouch can have the shape of domes with flanges (semi-spherical shape) of different shapes and sizes that are anchored in the peripheral region of the burn by high-performance hydrogel glue to inhibit detachments and leaks (e.g., FIG. 2). In addition, FIGS. 3A-3F show various embodiments for the application of hydrodynamic dressing pouches as well as an example of a full-body container.

Certain embodiments of the subject invention provide systems or methods following, aligned with, or supporting the guidelines recommended by the AMERICAN BURN ASSOCIATION, including but not limited to the following. Adults and children with burns greater than 20% total body surface area (TBSA) should undergo formal fluid resuscitation using estimates based on body size and surface area burned. Common formulas used to initiate resuscitation estimate a crystalloid need for 2-4 ml/kg body weight/% TBSA during the first 24 hours. Fluid resuscitation, regardless of solution type or estimated need, should be titrated to maintain a urine output of approximately 0.5-1.0 ml/kg/hour in adults and 1.0-1.5 ml/kg/hour in children. Maintenance fluids should be administered to children in addition to their calculated fluid requirements caused by injury. Increased volume requirements can be anticipated in patients with full-thickness injuries, inhalation injury and a delay in resuscitation.

Turning now to the figures, FIG. 1 Schematically illustrates a Hydrodynamic Dressing Hand and Arm Pouch adapted to treat a burn on or near a hand according to an embodiment of the subject invention. In this embodiment, a sealing adhesive stripe seals around the patient's forearm above a treatment fluid inlet configured to supply fluid to an inlet distribution ring contained within the plastic pouch. Fluid can additionally be drawn from the pouch and recirculated through a high-flow user-directable nozzle, allowing a practitioner to hydrodynamically debride the wound bed without needing to remove the Hydrodynamic Dressing itself. The High-Flow Debridement Nozzle can be a separate physical object which accesses the wound through a specific orifice, or can be physically integrated into the pouch itself. Fluid continuously or intermittently drains from the pouch through a pressure-check valve, activated either manually, or by an internal pressure exceeding the pressure specification of the check valve.

FIG. 2 Schematically illustrates an Automated Fluid Delivery System (AFDS) connected to a Hand and Arm Hydrodynamic Dressing Pouch adapted to treat a burn on the ventral area of a patient according to an embodiment of the subject invention. In this embodiment, a plastic pouch encapsulates the burn site. Adhesive seal strips form a fluid barrier, sequestering the burn site and creating a pouch. Fluid is delivered to the pouch Inlet Distribution Ring via the Fluid Input by the Automatic Fluid Delivery System. Fluid continuously or intermittently drains from the pouch through a pressure-check valve, activated either manually, or by an internal pressure exceeding the pressure specification of the check valve. Fluid drawn from the pouch can be recirculated through a high-flow user-directable nozzle, allowing a practitioner to hydrodynamically debride the wound bed without needing to remove the Hydrodynamic Dressing itself. The High-Flow Debridement Nozzle can be a separate physical object which accesses the wound through a specific orifice, or can be physically integrated into the pouch itself.

FIGS. 3A-3F Schematically illustrate respective Hydrodynamic Dressing designs adapted to treat burns in specific areas of patient anatomy according to certain embodiments of the subject invention. In each respectively illustrated embodiment, Fluid is delivered to the Fluid Input(s), and continuously or intermittently drains from the Hydrodynamic Dressing through one or many pressure-check valve(s), activated either manually, or by an internal pressure exceeding the pressure specification of the check valve. Fluid outputs can be recirculated through a high-flow user-directable nozzle, allowing a practitioner to hydrodynamically debride the wound bed without needing to remove the Hydrodynamic Dressing itself. The provided High-Flow Debridement Nozzle can be a separate physical object which accesses the wound through one or many specific orifices, or can be physically integrated into the Hydrodynamic Dressing itself.

FIG. 3A Schematically illustrates a Hand and Arm Hydrodynamic Dressing Pouch design adapted to treat burns on the hand or wrist according to certain embodiments of the subject invention. In this embodiment, a flexible plastic pouch encapsulates the burn site. An adhesive seal strip forms a fluid barrier, sequestering the burn site within the pouch. Fluid is delivered to the pouch Inlet Distribution Ring via the Fluid Input by the Automatic Fluid Delivery System. Fluid continuously or intermittently drains from the pouch through a pressure-check valve, activated either manually, or by an internal pressure exceeding the pressure specification of the check valve. Fluid drawn from the pouch can be recirculated through a high-flow user-directable nozzle, allowing a practitioner to hydrodynamically debride the wound bed without needing to remove the Hydrodynamic Dressing itself. The High-Flow Debridement Nozzle can be a separate physical object which accesses the wound through a specific orifice, or can be physically integrated into the pouch itself.

FIG. 3B Schematically illustrates a Leg and/or Foot Hydrodynamic Dressing Pouch design adapted to treat burns on the leg or foot according to certain embodiments of the subject invention. In this embodiment, a plastic pouch encapsulates the burn site. Adhesive seal strips form a fluid barrier, sequestering the burn site and creating a pouch. Fluid is delivered to the pouch Inlet Distribution Ring via the Fluid Input by the Automatic Fluid Delivery System. Fluid continuously or intermittently drains from the pouch through a pressure-check valve, activated either manually, or by an internal pressure exceeding the pressure specification of the check valve. Fluid drawn from the pouch can be recirculated through a high-flow user-directable nozzle, allowing a practitioner to hydrodynamically debride the wound bed without needing to remove the Hydrodynamic Dressing itself. The High-Flow Debridement Nozzle can be a separate physical object which accesses the wound through a specific orifice, or can be physically integrated into the pouch itself.

FIG. 3C Schematically illustrates a Body and/or Torso Hydrodynamic Dressing Pouch design adapted to treat burns on the multiple regions of the body, or occurring on both ventral and dorsal side of a region, or both, according to certain embodiments of the subject invention. In this embodiment, a plastic pouch encapsulates one or more of the following regions of the body; chest, shoulders, upper arms, upper legs, neck, abdomen, hips, groin, and buttocks. Adhesive scal strips form a fluid barrier, sequestering the burn site and creating a pouch. Fluid is delivered to the pouch Inlet Distribution Ring via the Fluid Input by the Automatic Fluid Delivery System. Fluid continuously or intermittently drains from the pouch through one or more pressure-check valves, activated either manually, or by an internal pressure exceeding the pressure specification of the check valve. Fluid drawn from the pouch can be recirculated through a high-flow user-directable nozzle, allowing a practitioner to hydrodynamically debride the wound bed without needing to remove the Hydrodynamic Dressing itself. The High-Flow Debridement Nozzle can be a separate physical object which accesses the wound through a specific orifice, or can be physically integrated into the pouch itself.

FIG. 3D Schematically illustrates a Partial Immersion Hydrodynamic Dressing design adapted to treat burns on the head or neck according to certain embodiments of the subject invention. In this embodiment, a rigid enclosure encapsulates the head, neck, and shoulders. Adhesive Foam at the interface between the dressing and the skin form a fluid barrier, sequestering the burn site from the external environment. Fluid is delivered to the Inlet Distribution Ring via the Fluid Inputs at the top of the Hydrodynamic Dressing by the Automatic Fluid Delivery System. Fluid continuously or intermittently drains from the dressing through one or more pressure-check valves located at various points on the dressing. These are either activated manually, or by an internal pressure exceeding the pressure specification of the check valve. Fluid exiting the dressing can be recirculated through a high-flow user-directable nozzle, allowing a practitioner to hydrodynamically debride the wound bed without needing to remove the Hydrodynamic Dressing itself. The High-Flow Debridement Nozzle can be a separate physical object which accesses the wound through a specific orifice, or can be physically integrated into the pouch itself. Because the mouth and nose of the patient are immersed in the fluid, breathing air is provided via a cannula. As the eyes region is also submerged, goggles are expected to be used during treatment.

FIG. 3E Schematically illustrates a Full Immersion Hydrodynamic Dressing design adapted to treat high surface area burns of the body according to certain embodiments of the subject invention. In this embodiment, a rigid transparent plastic chamber holds the patient, immersed in fluid. Fluid is delivered to the Inlet Distribution Ring via the Fluid Inputs at the top of the Hydrodynamic Dressing by the Automatic Fluid Delivery System. Fluid continuously or intermittently drains from the dressing through one or more pressure-check valves located at various points on the dressing. These are either activated manually, or by an internal pressure exceeding the pressure specification of the check valve. Fluid exiting the dressing can be recirculated through a high-flow user-directable nozzle, allowing a practitioner to hydrodynamically debride the wound bed without needing to remove the Hydrodynamic Dressing itself. The High-Flow Debridement Nozzle can be a separate physical object which accesses the wound through a specific orifice, or can be physically integrated into the pouch itself.

FIG. 3F Schematically illustrates a set of Hydrodynamic Facial Pouch Dressing designs adapted to treat face and neck burns of the body according to certain embodiments of the subject invention. In this embodiment, a flexible plastic pouch encapsulates a portion of the head, face and/or neck. Adhesive seal strips form a fluid barrier, sequestering the burn site and creating a pouch. Fluid is delivered to the pouch Inlet Distribution Ring or directly into the pouch via the Fluid Input by the Automatic Fluid Delivery System. Fluid continuously or intermittently drains from the pouch through one or more pressure-check valves, activated either manually, or by an internal pressure exceeding the pressure specification of the check valve. Fluid drawn from the pouch can be recirculated through a high-flow user-directable nozzle, allowing a practitioner to hydrodynamically debride the wound bed without needing to remove the Hydrodynamic Dressing itself. The High-Flow Debridement Nozzle can be a separate physical object which accesses the wound through a specific orifice, or can be physically integrated into the pouch itself.

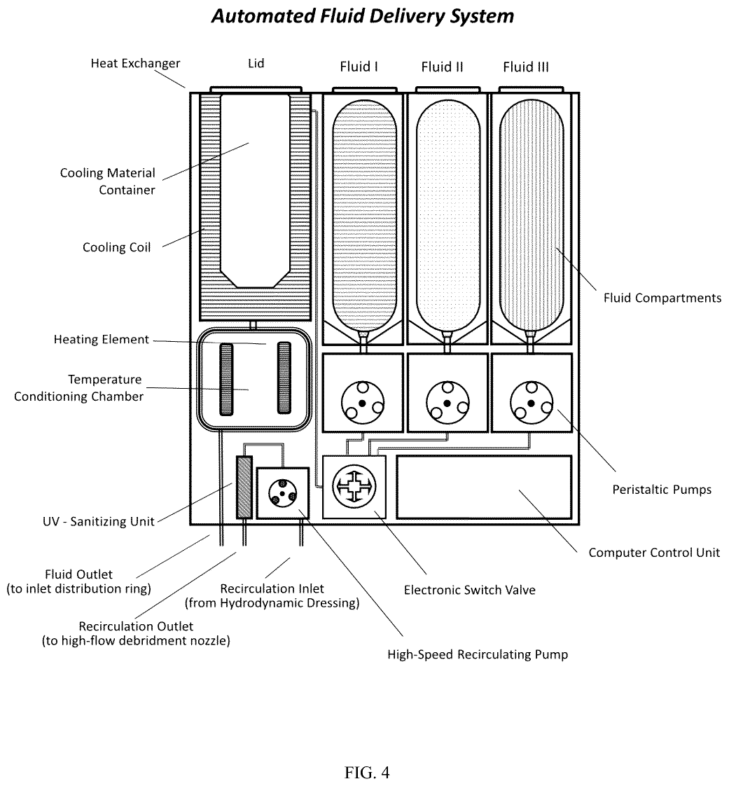

FIG. 4 Schematically illustrates an Automated Fluid Delivery System (AFDS) according to an embodiment of the subject invention. The Automatic Fluid Delivery System is controlled in its entirety by a Computer Control Unit. The AFDS has multiple compartments for holding treatment fluids, or packaged treatment fluids. Fluids are drawn individually by peristaltic pumps to a cooling chamber, containing a heat exchanger. Cooling power is provided by either water ice, dry ice, by an external chiller, or by an onboard chiller. Next, fluid flows to the Temperature Conditioning Chamber containing another heat exchanger. Heat is added to the fluid solution by resistive dissipation, after which it is outputted through the Fluid Outlet to the Hydrodynamic Dressing. Separately, fluid is recirculated from the dressing by a high-speed recirculation peristaltic pump, through a UV sanitizing unit, and out through the recirculation outlet. This system provides treatment fluids to the high-flow debridement nozzle.

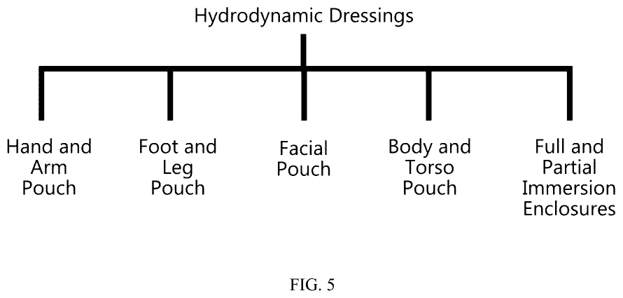

FIG. 5 Schematically illustrates a functional taxonomy organizing respective Hydrodynamic Dressing designs adapted to treat burns in specific areas of patient anatomy according to certain embodiments of the subject invention.

### Materials and Methods

Burns can be treated within the hydrodynamic pouch with a series of topical treatment irrigation fluids comprising Therapeutic Fluids that suffuse the pouch allowing various therapeutic actions, including but not limited to the following: intense hydration conducive to wound healing; restoration of osmotic pressure in the burned tissue; chemical and hydrodynamic micro-debridement and removal of bacterial biofilm; and inhibition of inflammation, edema, and abnormal fibrous tissue proliferation.

In certain embodiments, three therapeutic fluids can be used sequentially to support, facilitate, and align with the natural progressions of tissue burn repair processes. The fluid first applied (Fluid I) will keep the wound clean, hydrated, and in hydrostatic circumstances conducive to wound repair, in addition to eliminating dead, coagulated, and devitalized tissue from the burn through a micro-chemical hydrodynamic debridement. Fluid I will be able to decrease pain, eliminate bacterial biofilm formation, and sanitize the wound of harmful compounds from the fire and its fumes. It can also promote tissue perfusion to remove pro-inflammatory soluble components within the interstitial fluid generated by the edema. In addition, Fluid I can condition burn tissue to cycles of cold temperatures to reduce severe edema and inflammation. Fluid II will follow Fluid I. Fluid II can comprise a non-aggressive cell-friendly cleansing and moisturizing solution designed to remove flaking from burnt skin layers, restore the isotonic condition of the wound bed, limit biofilm establishment, promote a continuous state of anti-inflammatory action, and reduce pain and edema. Fluid III will follow Fluid II and will act in the process of epithelialization and wound closure as the wound granulation process advances, the inflammation subsides, and the first symptoms of epithelialization show. Fluid III, a bioactive preparation, can modulate myofibroblasts and hypertrophic chondrocytes' aberrant metabolism. Fluid III's primary purpose is to encourage high-quality tissue healing while minimizing keloids, hypertrophic scarring, and fibrosis.

Embodiments provide the following advantageously therapeutic fluid compositions and functions.

Therapeutic Fluid I can be administrated cold (12-15° C.) within 48-72 hours after burning trauma and has the following functions:
Removes coagulated/dead tissue resulting from the burn.
Performs a hydrodynamic micro-chemical debridement on the burn site.
Increases tissue perfusion to remove pro-inflammatory soluble components within the interstitial fluid formed with the edema.
Eradicates bacterial biofilm formation.
Performs the purification of toxic substances from the flames in the wound.
Reduces wound pain.

TABLE 1
Composition of Fluid I

| Ingredient Name | CAS Number | Concentration | Conc. Range |
|---|---|---|---|
| 1-dodecylazepan- | 59227-89-3 | 1.0% | 0.1-2.0% |
| 2-one | | | |
| Coco amido | 61789-40-0 | 2.0% | 0.1-4.0% |
| propyl betaine | | | |
| PHMB | 28757-47-3 | 0.05% | 0.001-0.100% |
| Sodium Heparin | 9041-08-1 | 200 UI/mL | 50-300 UI/mL |
| Gentamycin | 36889-17-5 | 0.005% | 0.001-0.01% |
| Sodium Benzoate | 532-32-1 | 0.5% | 0.1-1.0% |
| Pure Sterile Water | 7732-18-5 | quantum sufficit | n.a. |

Fluid I can be administrated in conjunction with venous hydration therapy for hydration to replace fluid and electrolyte losses, for example:
Pure 0.9% NaCl can be administered on day 1 (310 mOsm/L, 154 mEq/L Na+; 154 mEq/L Cl)—First 24 hs
On the second day, the 0.9% NaCl solution can be diluted with isotonic glucose solution (SGI) at 5% in the ratio of 1: SGI/1: 0.9% NaCl and equally administered over 24 hours. Add 10% KCl and 10% calcium gluconate, 2 mL and 1 mL, respectively, for each 100 mL of the maintenance solution. Consider the volume administered by the oral or enteral intake infusion pump.
Third-day Intravenous hydration repair formula: 1 mL×Weight×% SCQ+maintenance solution. The 0.9% NaCl solution can be diluted with SGI 5%, in the ratio 2: SGI/1: 0.9% NaCl and equally administered over 24 hours. Add KCl and calcium gluconate. Consider the oral or enteral intake through the infusion pump in calculating the volume administered.

Therapeutic Fluid II can be administrated at room temperature (15-25° C.) within 3-5days after burning trauma and has the following functions:
Provides a Cleansing Solution to remove flaking from burnt skin layers.
Performs a hydrodynamic micro-chemical debridement on the burn site.
Restores isotonic condition in the wound bed.
Promotes a topical anti-edematous and anti-inflammatory action.
Mitigates bacterial biofilm growth.
Reduces wound pain.

TABLE 2
Composition of Fluid II

| | CAS | | |
|---|---|---|---|
| Ingredient Name | Number | Concentration | Conc. Range |
| Epigallocatechin | 989-51-5 | 0.7% | 0.05-3.0% |
| Gallate (EGCG) | | | |
| Coco amido propyl | 61789-40-0 | 2.0% | 0.1-4.0% |
| betaine | | | |
| Glycerol | 56-81-5 | 3.0% | 0.1-3.0% |
| PHMB | 28757-47-3 | 0.05% | 0.001-0.100% |
| Sodium Heparin | 9041-08-1 | 200 UI/mL | 50-300 UI/ml |
| Gentamycin | 36889-17-5 | 0.005% | 0.001-0.01% |
| Sodium Benzoate | 532-32-1 | 0.5 | 0.1-1.0% |
| EDTA | 60-00-4 | 0.55% | 0.05-1.0% |
| Phosphate Buffer | 7558-79-4 | 0.1M | n.a. |
| 0.1M pH 8.0 | | | |
| Pure Sterile Water USP | 7732-18-5 | quantum | n.a. |
| | | sufficit | |

Therapeutic Fluid III can be administrated at a temperature (25-30° C.) within 6-9days after burning trauma and has the following functions:
Inhibitor of the conversion of myofibroblasts and hypertrophic chondrocytes.
Promote the reduction of hypertrophic scars, keloids, and fibrosis.
Promote a more homogeneous wound healing without stains and roughness.
Promote the reduction of contractures and loss of fine motor movement.
Promotes a topical anti-edematous and anti-inflammatory action.
Mitigates bacterial biofilm growth.

TABLE 3
Composition of Fluid III

| | | Concentration | Conc. |
|---|---|---|---|
| Ingredient Name | CAS Number | (%) | Range (%) |
| Poloxamer | 9003-11-6 | 1.5% | 0.1-2.0% |
| Coco amido propyl | 61789-40-0 | 2.0% | 0.1-4.0% |
| betaine | | | |
| Glycerol | 56-81-5 | 3.0% | 0.1-3.0% |
| PHMB | 28757-47-3 | 0.05% | 0.001-0.100% |
| Sodium Heparin | 9041-08-1 | 300 UI/mL | 50-300 UI/mL |
| Gentamycin | 36889-17-5 | 0.005% | 0.001-0.01% |
| Sodium Benzoate | 532-32-1 | 0.5% | 0.1-1.0% |
| EDTA | 60-00-4 | 1.0% | 0.05-2.0% |
| Polyaspartic Acid | 25608-40-6 | 1.0% | 0.5-3.0% |
| FIACE | 518-82-1 | 2.0% | 0.2-4.0% |
| Emodin, Naringenin, | 480-41-1 | | |
| Hesperidin, Astragalin | 520-26-3 | | |
| | 480-10-4 | | |
| FLACTA | 520-18-3 | 2.0% | 0.2-4.0% |
| Kaempferol, Quercetin, | 117-39-5 | | |
| Genistein, Apigenin, | 529-59-9 | | |
| Myricetin | 520-36-5 | | |
| | 529-44-2 | | |
| Trolox | 53188-07-1 | 0.07% | 0.01-0.5% |
| Phenoxyethanol | 122-99-6 | 0.7% | 0.1-1.5% |
| Pure Sterile Water USP | 7732-18-5 | quantum | n.a. |
| | | sufficit | |

TABLE 4
Selected Ingredients and Functions

| Ingredients | Function |
|---|---|
| 1-dodecylazepan-2- | Azone (1-dodecylazacycloheptan-2-one) is an agent that has been |
| one (Azone) | shown to enhance the percutaneous absorption of drugs. Azone is |
| (CAS: 59227-89-3) | thought to act by partitioning into skin lipid bilayers, thereby |
| | disrupting the structure. Absorption enhancers are substances used for |
| | temporarily increasing a membrane's permeability (e.g., the skin and |
| | mucosa), either by interacting with its components (lipids or proteins) |
| | or by increasing the membrane/vehicle partition coefficient. The use |
| | of Azone in this solution is intended to increase the permeability of |
| | sodium heparin in inflamed and coagulated tissues in the burn region, |
| | increasing tissue permeability and, consequently, osmotic perfusion. |
| Cocamidopropyl | Cocamidopropyl betaine is a mixture of closely related organic |
| betaine | compounds derived from coconut oil and dimethylaminopropylamine |
| (CAS: 61789-40-0) | that typically acts as an amphoteric surfactant in cosmetics and |
| | personal care products. It is a zwitterionic ammonium compound and |
| | fatty acid amide that contains a long hydrocarbon chain and a polar |
| | group at each end. Cocamidopropyl betaine is used as a foam booster |
| | in shampoos, emulsifying agent, a thickener, an antistatic agent, and |
| | rarely an antiseptic agent. Impurities formed during the manufacturing |
| | process are thought to increase the prevalence of contact sensitization |
| | and mild skin irritations. Cocamidopropyl betaine is employed to |
| | solubilize and solvate organic compounds, cellular debris from |
| | burning skin, and biological fluid secretions from damaged and |
| | inflamed tissue. |
| Polyhexamethylene | Polyhexamethylene biguanide (PHMB; polyhexanide) is a broad- |
| biguanide (PHMB; | spectrum antimicrobial biocide that kills bacteria, fungi, parasites, and |
| polyhexanide) | certain viruses with a high therapeutic index; it is widely used in |
| (CAS: 28757-47-3) | clinics, homes, and industries. PHMB is most commonly used as a |
| | biocide but is also an important drug used in several topical |
| | applications. PHMB is composed of repeating basic biguanide units |
| | connected by hexamethylene hydrocarbon chains, providing a cationic |
| | and amphipathic structure. Despite extensive use over several decades |
| | and efforts to identify acquired resistant mutants, resistance to PHMB |
| | has not been reported. PHMB present in Fluid I has the biocidal |
| | function of eliminating opportunistic microorganisms that infect the |
| | burn after the injury. In addition, it mitigates the growth of bacteria in |
| | the fluid phase of the device. |
| Heparin Sodium | Heparin sodium is a glycosaminoglycan anticoagulant that binds to |
| (CAS: 9041-08-1) | antithrombin III to form a heparin-antithrombin III complex. The |
| | complex binds to and irreversibly inactivates thrombin and other |
| | activated clotting factors IX, X, XI, and XII and inhibits the |
| | transformation of fibrinogen to fibrin. Heparin in all Fluids is an active |
| | anticoagulant that dissolves blood clots on the skin's surface. It helps |
| | to dissolve the blood clot and reduce the pain and swelling due to |
| | inflammation and coagulation caused by the burn injury. In addition, |
| | it increases tissue permeability to help tissue perfusion and the |
| | reduction of edemas and increases skin permeability allowing other |
| | drugs to diffuse better and faster into the skin. Heparin promotes the |
| | microcirculatory-modulatory action determining important control of |
| | the microcirculation in case of excessive vasoconstriction or |
| | vasodilatation. |
| Gentamicin X2 | A broad-spectrum aminoglycoside antibiotic produced by |
| (CAS: 36889-17-5) | fermentation of Micromonospora purpurea or M. echinospora. |
| | Gentamicin is an antibiotic complex consisting of four major (C1, C1a, |
| | C2, and C2a) and several minor components. This agent irreversibly |
| | binds to the bacterial 30S ribosomal subunit. Specifically, this |
| | antibiotic is lodged between 16S rRNA and S12 protein within the 30S |
| | subunit. This leads to interference with translational initiation |
| | complex, misreading of mRNA, thereby hampering protein synthesis |
| | and resulting in bactericidal effect. Aminoglycosides are mostly |
| | ineffective against anaerobic bacteria, fungi and viruses. Gentamicin |
| | in Fluid I and II is active against a wide range of bacterial infections, |
| | mostly Gram-negative bacteria including Pseudomonas, Proteus, |
| | Citrobacter, Enterobacter, Escherichia coli, Klebsiella pneumoniae, |
| | Enterobacter aerogenes, Serratia, and the Gram-positive |
| | Staphylococcus. Gentamicin is also used to inhibit contamination of |
| | the sterile Fluids. Gentamicin is one of the few heat-stable antibiotics |
| | that remain active even after autoclaving, which makes it particularly |
| | useful in the preparation of sterile therapeutic fluids. |
| Sodium Benzoate | Sodium benzoate is an organic sodium salt resulting from the |
| (CAS: 532-32-1) | replacement of the proton from the carboxy group of benzoic acid by |
| | a sodium ion. It has a role as an antimicrobial food preservative, a drug |
| | allergen, an EC 1.13.11.33 (arachidonate 15-lipoxygenase) inhibitor, |
| | an EC 3.1.1.3 (triacylglycerol lipase) inhibitor, an algal metabolite, a |
| | human xenobiotic metabolite and a plant metabolite. Sodium benzoate |
| | is used as a preservative in the Fluids. |
| Glycerol | Glycerol is used as a solvent, preservative, anti-freezing agent, |
| (CAS: 56-81-5) | thickening agent, and humectant. It is used in the preparation of |
| | pharmaceutical and personal care products, thereby improving |
| | smoothness and providing lubrication. The glycerin in Fluid II and III |
| | has the function of reducing and controlling the internal osmotic |
| | pressure of the burn tissues. It also has the function of stabilizing |
| | macromolecules such as proteins and proteoglycans. |
| Poloxamer | Poloxamers are defined as polyoxyethylene, polyoxypropylene blocks |
| (CAS: 9003-11-6) | of synthetic polymers. Poloxamer is a wound cleaning agent that can |
| | manage the elimination of exudate, slough, necrotic debris, and |
| | associated microbial contaminants, toxins, matrix metalloproteinases |
| | (MMPs), and cytokines as well as dressing residue without adversely |
| | impacting cellular activity vital to the wound healing process or |
| | colonizing the underlying tissue with microorganisms and detached |
| | biofilm. Poloxamer in Fluid III increases the solubility and stability of |
| | flavonoid compounds in the water phase. |
| FLACTA Solution | FLACTA is a proprietary optimized blend of flavonoid designated to |
| (Flavonoid Calcium | effectively reduce antioxidant activity during wound healing and |
| Trappers Activator) | activate the uptake of calcium molecules by the mitochondria. Wound |
| | healing is mostly related to the antioxidant activity of the chemical |
| | agent used; antioxidants significantly accelerate wound healing by |
| | removing the free oxygen radicals and increasing colloid synthesis. It |
| | has potent anti-inflammatory and antioxidant action and regulates the |
| | conversion of skin fibroblasts into myofibroblasts through |
| | sequestration of calcium and reactive oxygen species. FLACTA |
| | Solution is composed of the following flavonoids: Kaempferol (CAS: |
| | 520-18-3), Quercetin (CAS: 117-39-5), Genistein (CAS: 529-59-9), |
| | Apigenin (CAS: 520-36-5) and Myricetin (CAS: 529-44-2). |
| FIACE Solution | FIACE is a flavonoid proprietary solution formulated to modulate |
| (Flavonoid Inhibitor | angiotensin converting enzyme-2 activity to suppress abnormal |
| of Angiotensin | collagen deposition by the myofibroblasts. One of the multiple |
| Converting Enzyme) | functions of the renin-angiotensin system (RAS) is known to be the |
| | activation of transforming growth factor beta (TGF-β1). TGF-β1- |
| | Smad2/3 is the major signaling pathway of fibrosis, which is |
| | characterized by the excessive production and accumulation of |
| | extracellular matrix (ECM) components, including collagen. Although |
| | the extracellular matrix (ECM) is an essential component of skin, skin |
| | fibrosis is characterized by the excessive production and accumulation |
| | of collagen and other ECM components, resulting in cellular |
| | dysfunction and the loss of tissue architecture, eventually leading to |
| | deformities. FIACE solution is composed of several antioxidant and |
| | anti-inflammatory flavonoids that modulate ACE-2 activity and Nitro |
| | Oxide (NO) production, such as: Emodin (CAS: 518-82-1), |
| | Naringenin (CAS: 480-41-1), Hesperidin (CAS: 520-26-3) and |
| | Astragalin (CAS: 480-10-4). Emodin is an anthraquinone derivative |
| | that occurs in many herbs, such as Rheum palmatum, Polygonum |
| | cuspidatum, and Polygonum multiflorum. Emodin possesses various |
| | pharmacological properties, including anticancer, hepatoprotective, |
| | anti-inflammatory, antioxidant, and antimicrobial activities. |
| | Naringenin is a flavonoid belonging to flavanones subclass. It is |
| | widely distributed in several Citrus fruits, bergamot, tomatoes and |
| | other fruits, being also found in its glycosides form (mainly naringin). |
| | Several biological activities have been ascribed to this phytochemical, |
| | among them antioxidant, antitumor, antiviral, antibacterial, anti- |
| | inflammatory, antiadipogenic and cardioprotective effects. Naringenin |
| | is endowed with broad biological effects on human health, which |
| | includes a decrease in lipid peroxidation biomarkers and protein |
| | carbonylation, promotes carbohydrate metabolism, increases |
| | antioxidant defenses, scavenges reactive oxygen species, modulates |
| | immune system activity, and also exerts anti-atherogenic and anti- |
| | inflammatory effects. It promotes the inhibition of hypertrophic scars |
| | by downregulating of TNF-α, IL-1β, IL-6 and TGF-β1. Decreases |
| | secretion of TGF-β1 and accumulation of intracellular TGF-β1 and the |
| | inhibition of TGF-β1 transport from the trans-Golgi network, and PKC |
| | activity. Hesperidin is an antioxidant flavonoid that modulate |
| | fibroblast nitro oxide (NO) synthesis, inhibits free-radicals (LOO) |
| | activity, and suppress the pro-inflammatory expression of NF-KB and |
| | COX-2. Topical application of hesperidin increases the regenerating |
| | capacity of wounds and reduces tissue damaging by excessive free- |
| | radicals oxidation. Astragalin is an antioxidant and anti-inflammatory |
| | flavonoid for the treatment of acute inflammation resulting from tissue |
| | injury. It suppresses the inflammatory signaling in the inflamed tissue |
| | as exhibited by the decreased myeloperoxidase activity and the |
| | decreased protein and transcriptional level of pro-inflammatory |
| | cytokines, including tumor necrosis factor-alpha, interleukin-1 beta, |
| | and interleukin-6. Moreover, it downregulates inducible nitric oxide |
| | synthase and cyclooxygenase-2 expressions and their pro- |
| | inflammatory products (nitric oxide and prostaglandin E2). |
| | Additionally, astragalin can decrease monocyte chemoattractant |
| | protein-1 and nuclear factor kappa B expression in the inflamed tissue. |
| Trolox | Trolox (6-hydroxy-2, 5, 7, 8-tetramethyl-chroman-2-carboxylic acid) |
| (CAS: 53188-07-1) | is a water-soluble analog of vitamin E with excellent antioxidant |
| | capacity for free radical scavenging. This compound can scavenge |
| | peroxy radicals eight times better than vitamin E in surfactant micelle. |
| | Transforming growth factor-b(TGF-b) as the major profibrogenic |
| | master cytokine, which in concert with other growth factors, promotes |
| | transdifferentiation of fibroblast into myofibroblasts. The numerical |
| | expansion of myofibroblasts promotes the stimulation of matrix gene |
| | expression, downregulation of matrix degradation, and induction of |
| | cellular apoptosis. TGF-b stimulates the production of reactive oxygen |
| | species (ROS) in various types of cells, whereas ROS activates TGF- |
| | b1 and mediates many of its fibrogenic effects. Therefore, antioxidant |
| | therapy can be very useful in the treatment of fibrosis. |
| Epigallocatechin | Epigallocatechin Gallate (EGCG) has several important properties that |
| Gallate (EGCG) | favor effective wound healing, such as anti-inflammatory, antioxidant, |
| (CAS: 989-51-5) | anti-microbial, and anti-fibrotic. EGCG inhibits the generation of |
| | certain pro-inflammatory cytokines released during the inflammatory |
| | phase of burn healing, such as TNFα, IL-1β, and IL-8, by |
| | downregulating tissue gene expressions. Reactive oxygen species |
| | (ROS) exert adverse effects on cells and tissues. Generally, low ROS |
| | levels are conducive to the activation of cell signaling pathways and |
| | angiogenesis, whereas high ROS levels induce oxidative stress and |
| | compromise tissue repair, leading to chronic nonhealing wounds |
| | accompanied by inflammation. The antioxidant effect of EGCG, as a |
| | bioactive component with antioxidants/free radical scavenger activity, |
| | can protect tissues from oxidative damage. EGCG inhibits the growth |
| | of bacteria in various ways, including disrupting cell membranes |
| | through interacting with surface proteins, decomposing essential |
| | metabolites, inhibiting relevant enzymes, inducing ROS stress, |
| | changing cell-wall structure, detaching cytoplasm, and inducing ROS |
| | stress. EGCG inhibits the growth of Escherichia coli, Pseudomonas |
| | aeruginosa, and Staphylococcus aureus. Moreover, EGCG interferes |
| | with the assembly of amyloid, which fibers from curli the formation |
| | subunits, and the generation of phosphoethanolamine-modified |
| | cellulose of fibrils, which impedes the formation of biofilms. EGCG |
| | has antifibrotic properties, as demonstrated using the model of human- |
| | derived keloid fibroblasts transplanted onto nude mice. The |
| | productions of collagen and keloids were reduced under EGCG |
| | treatment. EGCG suppresses the pathological characteristics of |
| | keloids by inhibiting the STAT3 signaling pathway. Also, the keloid |
| | organ culture model for evaluating the antifibrotic effect of EGCG |
| | treatment shows the decreased size of the keloid, suppresses |
| | intrakeloid proliferation, reduces collagen production, and |
| | downregulates the transcription of major fibrosis-related pathways, |
| | including VEGF, matrix metalloproteinases (MMP-2 and -9) and |
| | TGF-β2. |
| Polyaspartic Acid | Polyaspartic acid (PASA) is a biodegradable, water-soluble |
| (CAS: 25608-40-6) | condensation polymer based on the amino acid aspartic acid that has |
| | strong affinity with Calcium and metal ions. It is a biodegradable |
| | calcium trapper to inhibit the conversion of myofibroblasts. |
| Ethylenediamine- | EDTA is a sequester (bind or confine) metal ions such as Ca, Mg, Ba |
| tetra acetic acid | in aqueous solution. It is used to chelate calcium ions in solution to |
| disodium salt | deplete the ion bioavailability for the conversion of myofibroblasts |
| (EDTA) | from fibroblasts |
| (CAS: 60-00-4) | |
| Phenoxyethanol | Phenoxyethanol is a common preservative in dermatological, cosmetic |
| (CAS: 122-99-6) | and vaccine formulations. It is a colorless oily liquid. It can be |
| | classified as a glycol ether and a phenol ether. The antimicrobial action |
| | of phenoxyethanol in Gram-negative bacteria is reported to be due to |
| | the disruption of cell membrane integrity and uncoupling of oxidative |
| | phosphorylation. Phenoxyethanol has germicidal and germistatic |
| | properties and it is effective against gram-negative and gram-positive |
| | bacteria, and the yeast Candida albicans. Phenoxyethanol is the major |
| | preservative of Fluid III. |

Absent specific direction to the contrary, the following substitutions or replacements are contemplated within the scope of the subject invention.

### Azone

1-geranylazacycloheptan-2-one (GACH)

### Cocamidopropyl Betaine

almondamidopropyl betaine, apricotamidopropyl betaine, avocadamidopropyl betaine, babassuamidopropyl, betaine, behenamidopropyl betaine, canolamidopropyl betaine, capryl/capramidopropyl betaine, coco/oleamidopropyl betaine, coco/sunfloweramidopropyl betaine, cupuassuaidopropyl betaine, isostearmidopropyl betaine, lauramidopropyl betaine, meadowfoamamidopropyl betaine, milkamidopropyl, betaine, minkamidopropyl betaine, myristamidopropyl betaine, oatamidopropyl betaine, oleamidopropyl betaine, olivamidopropyl betaine, palmamidopropyl betaine, palmitamidopropyl betaine, palm kernelamiodpropyl betaine, ricinoleamidopropyl betaine, sesamidopropyl betaine, shea butteramidopropyl, betaine, soyamidopropyl betaine, stearamidopropyl betaine, betaine, tallowamidopropyl undecyleneamidopropyl betaine, wheat germamidopropyl betaine, dimethicone propyl PG-Betaine, Cocamidopropyl hydroxysultaine, formamindopropyl betaine.

### PHMB

### 6-Diguanidinohexane,

### Sodium Benzoate

Potassium benzoate, sodium sorbate, potassium sorbate, Phenoxyethanol, Caprylylglycol

### Glycerol

### Glycerine, Sorbitol, Mannitol, Xylitol

### Trolox

Alpha-tocopherol, alpha-tocopherol acetate

### EDTA

Ethylene glycol-bis(2-aminoethyl)-tetra acetic ac (EGTA), Methylgycine diacetic acid (MGDA)

Embodiments of the subject invention provide an Automated Fluid Delivery System that delivers conditioned Therapeutic Fluids to hydrodynamic dressing pouches and can include the following essential components (e.g., as shown in FIG. 4):

Embodiments provide Reservoir Drawers that can be stainless steel independent cassettes (e.g., 304 or 316 stainless) that are resistant to oxidation and that can contain one or more sterile Therapeutic Fluid containers. The drawers can have an opening with a lid at the top for inserting containers, a window at the front for observing the fluid level, and a sterile tube with a quick-sanitary connection at the bottom for connecting the respective fluids. Embodiments provide a drain outlet on the back to facilitate cleaning and sanitization.

Embodiments provide Peristaltic Control Unit and Pumps. The provided peristaltic control unit is the programmable electronic center of the entire system, in certain embodiments providing a touchscreen monitor where the entire setting of the peristaltic pumps, switch valve, and temperature control of the device can be adjusted. Certain embodiments provide three peristaltic pumps, each respectively connected to a different fluid container. These pumps are controlled by a peristaltic control unit capable of pumping each fluid independently with a flow range from 1 to 100 mL/min. The tubing connecting the reservoirs to the peristaltic pumps can be made of medical-grade autoclavable silicone, which can be replaced by opening the front access of the equipment. These tubes can be coupled to an electronic switch valve that selects and connects each different line at the inlet of the fluid temperature control system.

Embodiments provide a Temperature Control System including a cooling coil, heater, thermostat, and conditioning chamber that adjusts the fluid temperature (e.g., between 10 to 30° C.) The fluid coming from one or more of the peristaltic pumps first enters the cooling coil and goes to the temperature conditioning chamber, where the temperature can be adjusted according to the programmed temperature and flow. In certain embodiments the cooling coil consists of a 316 stainless steel spiral tube inserted in an aluminum block with thermal insulation with a cylindrical cavity in the central part to insert cooling material (dry ice). The conditioning chamber can be a 316 stainless-steel heat exchanger or container coated with thermo-insulating material, a heating element, and a thermostat coupled internally for temperature control.

Embodiments provide a High-Speed Debridement Recirculation System, wherein a high-speed recirculation pump provides hydrodynamic debridement inside the dressing pouch through high-speed peristaltic pumping of the fluid inside the pouch to recirculate through an ultraviolet light sterilization until it returns to the dressing pouch through a directional jet nozzle. This high-speed recirculating flow can be applied to perform hydrodynamic debridement.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

The invention may be better understood by reference to certain illustrative examples, including but not limited to the following:

Embodiment 1. A system for treating a burn wound caused by a burning trauma, the system comprising:
a Hydrodynamic Dressing (HD) configured and adapted to enclose the burn wound;
three distinct Therapeutic Fluids (TFs), each respectively formulated to support a designated stage of healing with respect to the burn wound; and
an Automated Fluid Delivery System (AFDS) configured and adapted to deliver each of the three TFs, respectively, to the burn wound enclosed within the HD at a respectively specified time, temperature, pressure, and flow rate according to a burn wound treatment protocol.

Embodiment 2. The system according to Embodiment 1, wherein the HD comprises a wound enclosure that isolates the wound from the nosocomial environment, facilitates wound observation, and facilitates hydrodynamic debridement.

Embodiment 3. The system according to Embodiment 2, wherein the AFDS comprises an automated conditioning and pumping unit that is configured and adapted to deliver each of the three TFs, respectively, to the burn wound enclosed within the HD in sequence, at specified flow rates, pressures, and temperatures.

Embodiment 4. The system according to Embodiment 3, wherein the HD comprises a nozzle configured and adapted to deliver one or more of the three distinct TFs at a measured rate to facilitate the removal of toxins and necrotic byproducts from the burn wound.

Embodiment 5. The system according to Embodiment 4, wherein the HD comprises a distributed array of nozzles, each respective nozzle configured and adapted to deliver one or more of the three distinct TFs at a measured rate to facilitate the removal of toxins and necrotic byproducts from the burn wound.

Embodiment 6. The system according to Embodiment 5, wherein the HD comprises an outlet port configurable to route fluids that pass across the burn wound enclosed within the HD to either a waste receptacle or a recirculating pump.

Embodiment 7. The system according to Embodiment 3, wherein the three distinct TFs comprise:
a first TF formulated to generate an optimal wound healing environment within 0 to 3 days after burning trauma;
a second TF formulated to generate an optimal wound healing environment within 3 to 5 days after burning trauma; and
a third TF formulated to generate an optimal wound healing environment within 6 to 12 days after burning trauma.

Embodiment 8. The system according to Embodiment 7, wherein a combination of the first TF, the second TF, and the third TF is formulated to provide intense hydration conducive to wound healing, to restore normal osmotic pressure in the burned tissue, to chemically and hydrodynamically micro-debride and flush away necrotic byproducts and oxidizing compounds, to remove bacterial biofilm, to maintain a hygienic wound environment, and to inhibit one or more undesirable effects selected from the list consisting of inflammation, edema, and excessive fibrous tissue proliferation.

Embodiment 9. The system according to Embodiment 8, wherein:
the first TF is formulated to:
   remove coagulated and dead tissue from the burn wound,
   perform hydrodynamic micro-chemical debridement on the burn wound,
   increase tissue perfusion to remove pro-inflammatory soluble components within an interstitial fluid formed with an edema on the burn wound,
   mitigate bacterial biofilm formation on the burn wound,
   purify toxic substances from the burn wound, and
   reduce pain from the burn wound;
the second TF is formulated to:
   remove flaking from burnt skin layers on the burn wound,
   perform hydrodynamic micro-chemical debridement on the burn wound,
   restore isotonic condition in the burn wound.
   promote a topical anti-edematous and anti-inflammatory action on the burn wound.
   mitigate bacterial biofilm growth on the burn wound, and
   reduce pain from the burn wound; and
the third TF is formulated to:
   modulate the activation and conversion of resident skin fibroblasts into myofibroblasts in response to burn injury,
   promote a reduction of hypertrophic scars, keloids, and fibrosis on the burn wound,
   promote a more homogeneous wound healing without stains and roughness on the burn wound,
   promote a reduction of contractures and loss of fine motor movement on the burn wound,
   promote a topical anti-edematous and anti-inflammatory action on the burn wound, and
   mitigate bacterial biofilm growth on the burn wound.

Embodiment 10. The system according to Embodiment 9, wherein the burn wound treatment protocol comprises:
administration of the first TF between 12 to 15° C. and within 48 to 72 hours after burning trauma;
administration of the second TF between 15 to 25° C. and within 3 to 5 days after burning trauma; and
administration of the third TF between 25 to 30° C. and within 6 to 9 days after burning trauma.

Embodiment 11. The system according to Embodiment 10, wherein the burn wound treatment protocol comprises:
administering the first TF in conjunction with venous hydration therapy for hydration to replace fluid and electrolyte losses, following recommendations of the American Burn Association, comprising:
   administering formal fluid resuscitation at a rate of 2-4 (ml/kg body weight) divided by percent total body surface area (TBSA) burned during the first 24 hours; and
   titrating fluid resuscitation to maintain a urine output of approximately 0.5-1.0 ml/kg/hour in an adult patient over 17 years of age or 1.0-1.5 ml/kg/hour in a pediatric patient 17 years of age or younger.

Embodiment 12. A method of treating a burn wound caused by a burning trauma, the method comprising the following steps:
providing a Hydrodynamic Dressing Pouch (HD) configured and adapted to enclose the burn wound;
providing three distinct Therapeutic Fluids (TFs), each respectively formulated to support a designated stage of healing with respect to the burn wound;
providing an Automated Fluid Delivery System (AFDS) comprising a wound enclosure that isolates the wound from the nosocomial environment, facilitates wound observation, and facilitates hydrodynamic debridement; and
delivering each of the three TFs, respectively, to the burn wound enclosed within the HD at a respectively specified time, temperature, pressure, and flow rate according to a burn wound treatment protocol

Embodiment 13. The method according to Embodiment 12, wherein the step of delivering each of the three TFs, respectively, to the burn wound enclosed within the HD comprises bringing each of the three TFs, respectively and sequentially, to a respective specified pressure and temperature at a respective specified time within an automated conditioning and pumping unit before or during delivering each of the three TFs, respectively, to the burn wound enclosed within the HD.

Embodiment 14. The method according to Embodiment 13, comprising removal of toxins and necrotic byproducts from the burn wound by action of a nozzle on the burn wound enclosed within the HD.

Embodiment 15. The method according to Embodiment 13, wherein the three distinct TFs comprise:
a first TF formulated to generate an optimal wound healing environment within 0 to 3 days after burning trauma;
a second TF formulated to generate an optimal wound healing environment within 3 to 5 days after burning trauma; and
a third TF formulated to generate an optimal wound healing environment within 6 to 9 days after burning trauma.

Embodiment 16. The method according to Embodiment 15, wherein a combination of the first TF, the second TF, and the third TF is formulated to provide intense hydration conducive to wound healing, to restore normal osmotic pressure in the burned tissue, to chemically and hydrodynamically micro-debride and flush away necrotic byproducts and oxidizing compounds, to remove bacterial biofilm, to maintain a hygienic wound environment, and to inhibit one or more undesirable effects selected from the list consisting of inflammation, edema, and excessive fibrous tissue proliferation.

Embodiment 17. The method according to Embodiment 16, wherein:
the first TF is formulated to:
   remove coagulated and dead tissue from the burn wound,
   perform hydrodynamic micro-chemical debridement on the burn wound,
   increase tissue perfusion to remove pro-inflammatory soluble components within an interstitial fluid formed with an edema on the burn wound,
   mitigate bacterial biofilm formation on the burn wound,
   purify toxic substances from the burn wound, and
   reduce pain from the burn wound;
the second TF is formulated to:
   remove flaking from burnt skin layers on the burn wound,
   perform hydrodynamic micro-chemical debridement on the burn wound,
   restore isotonic condition in the burn wound.
   promote a topical anti-edematous and anti-inflammatory action on the burn wound.
   mitigate bacterial biofilm growth on the burn wound, and
   reduce pain from the burn wound; and
the third TF is formulated to:
   modulate the activation and conversion of resident skin fibroblasts into myofibroblasts in response to burn injury,
   promote a reduction of hypertrophic scars, keloids, and fibrosis on the burn wound,
   promote a more homogeneous wound healing without stains and roughness on the burn wound,
   promote a reduction of contractures and loss of fine motor movement on the burn wound,
   promote a topical anti-edematous and anti-inflammatory action on the burn wound, and
   mitigate bacterial biofilm growth on the burn wound.

Embodiment 18. The method according to Embodiment 17, wherein the burn wound treatment protocol comprises:
administration of the first TF between 12 to 15° C. and within 48 to 72 hours after burning trauma;
administration of the second TF between 15 to 25° C. and within 3 to 5 days after burning trauma; and
administration of the third TF between 25 to 30° C. and within 6 to 9 days after burning trauma.

Embodiment 19. The method according to Embodiment 18, wherein the burn wound treatment protocol comprises:
administering the first TF in conjunction with venous hydration therapy for hydration to replace fluid and electrolyte losses, following recommendations of the American Burn Association, comprising:
administering formal fluid resuscitation at a rate of 2-4 (ml/kg body weight) divided by percent total body surface area (TBSA) burned during the first 24 hours; and
titrating fluid resuscitation to maintain a urine output of approximately 0.5-1.0 ml/kg/hour in an adult patient over 17 years of age or 1.0-1.5 ml/kg/hour in a pediatric patient 17 years of age or younger.

Embodiment 20. A system for treating a burn wound caused by a burning trauma, the system comprising:
a Hydrodynamic Dressing Pouch (HD) configured and adapted to enclose the burn wound;
three distinct Therapeutic Fluids (TFs), each respectively formulated to support a designated stage of healing with respect to the burn wound; and
an Automated Fluid Delivery System (AFDS) configured and adapted to deliver each of the three TFs, respectively, to the burn wound enclosed within the HD at a respectively specified time, temperature, pressure, and flow rate according to a burn wound treatment protocol;
wherein the HD comprises a wound enclosure that isolates the wound from the nosocomial environment, facilitates wound observation, and facilitates hydrodynamic debridement;
wherein the AFDS comprises an automated conditioning and pumping unit that is configured and adapted to deliver each of the three TFs, respectively, to the burn wound enclosed within the HD in sequence, at specified flow rates, pressures, and temperatures;
wherein the HD comprises a distributed array of nozzles, each respective nozzle configured and adapted to deliver one or more of the three distinct TFs at a measured rate to facilitate the removal of toxins and necrotic byproducts from the burn wound;
wherein the HD comprises an outlet port configurable to route fluids that pass across the burn wound enclosed within the HD to either a waste receptacle or a recirculating pump;
wherein the three distinct TFs comprise:
   a first TF formulated to generate an optimal wound healing environment within 0 to 3 days after burning trauma;
   a second TF formulated to generate an optimal wound healing environment within 3 to 5 days after burning trauma; and
   a third TF formulated to generate an optimal wound healing environment within 6 to 9 days after burning trauma;
wherein a combination of the first TF, the second TF, and the third TF is formulated to provide intense hydration conducive to wound healing, to restore normal osmotic pressure in the burned tissue, to chemically and hydrodynamically micro-debride and flush away necrotic byproducts and oxidizing compounds, to remove bacterial biofilm, to maintain a hygienic wound environment, and to inhibit one or more undesirable effects selected from the list consisting of inflammation, edema, and excessive fibrous tissue proliferation;
wherein the first TF is formulated to:
   remove coagulated and dead tissue from the burn wound,
   perform hydrodynamic micro-chemical debridement on the burn wound,
   increase tissue perfusion to remove pro-inflammatory soluble components within an interstitial fluid formed with an edema on the burn wound,
   mitigate bacterial biofilm formation on the burn wound,
   purify toxic substances from the burn wound, and
   reduce pain from the burn wound;
wherein the second TF is formulated to:
   remove flaking from burnt skin layers on the burn wound,
   perform hydrodynamic micro-chemical debridement on the burn wound,
   restore isotonic condition in the burn wound.
   promote a topical anti-edematous and anti-inflammatory action on the burn wound.
   mitigate bacterial biofilm growth on the burn wound, and
   reduce pain from the burn wound; and
wherein the third TF is formulated to:
   modulate the activation and conversion of resident skin fibroblasts into myofibroblasts in response to burn,
   promote a reduction of hypertrophic scars, keloids, and fibrosis on the burn wound,
   promote a more homogeneous wound healing without stains and roughness on the burn wound,
   promote a reduction of contractures and loss of fine motor movement on the burn wound,
   promote a topical anti-edematous and anti-inflammatory action on the burn wound, and
   mitigate bacterial biofilm growth on the burn wound;
wherein the burn wound treatment protocol comprises:
   administration of the first TF between 12 to 15° C. and within 48 to 72 hours after burning trauma;
   administration of the second TF between 15 to 25° C. and within 3 to 5 days after burning trauma; and
   administration of the third TF between 25 to 30° C. and within 6 to 9 days after burning trauma; and
wherein the burn wound treatment protocol comprises administering the first TF in conjunction with venous hydration therapy for hydration to replace fluid and electrolyte losses, comprising:
administering formal fluid resuscitation at a rate of 2-4 (ml/kg body weight) divided by percent total body surface area (TBSA) burned during the first 24 hours; and
titrating fluid resuscitation to maintain a urine output of approximately 0.5-1.0 ml/kg/hour in an adult patient over 17 years of age or 1.0-1.5 ml/kg/hour in a pediatric patient 17 years of age or younger.

Although the present invention has been illustrated and described herein with reference to preferred embodiments and specific examples thereof, it will be readily apparent to those of ordinary skill in the art that other embodiments and examples may perform similar functions and/or achieve like results.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### EXAMPLE 1—PROSPECTIVE DESCRIPTION OF TESTING VERIFICATIONS

While not being bound by theory, the inventors hypothesize that when evaluated utilizing a porcine burn wound model (or other suitable in vitro and in vivo models including large animal and small animal models) and later through human clinical studies, the application of one or more embodiments of the subject invention will reduce the formation of skin scarring, fibrosis, and contracture from burns. Furthermore, the inventors hypothesize that when evaluated in-vitro, Solutions II and III will inhibit the over-expression of myofibroblast conversion and reduce fibrous tissue layer deposition.

While not being bound by theory, the inventors hypothesize that Solutions I, II, and III will all inhibit the growth of bacteria and fungi in-vitro and in-vivo. Solutions I and II will prove to be inhibitive to the proliferation of bacterial biofilms via effective chemical debridement and destructive to benthic bacteria and biofilm extra-cellular matrix due to their debridement efficacy.

While not being bound by theory, the inventors hypothesize the following Prospective Description of Outcomes: Design of Experiments (DoE) experiments evaluating temperatures, pressures, and flow rates within the ranges described in Table A will improve and provide further reduction in scarring, fibrosis, and contracture. DoE studies conducted to evaluate optimal irrigation solution ingredient concentrations will have a similar effect when assessed in a porcine burn wound model. Furthermore, these and further DoE studies are expected to improve performance in terms of anti-microbial action and biocompatibility (e.g., cytotoxicity, systemic toxicity, pyrogenicity, acute toxicity) of irrigation solutions. See, for example, the compositions disclosed in Table 1 through Table 4, above, for contemplated ranges, compositions, ingredients, and concentrations of solutions.

TABLE 5
Selected Physical Process Parameters to be Optimized via DoE

| | Expected | Expected |
|---|---|---|
| | Minimum | Maximum |
| Wound Enclosure Pressure | 1 ATM | 2 ATM |
| Therapeutic Solution Temperature | 10° C. | 40° C. |
| Therapeutic Solution Flow Rate | 0.1 mL/min | 30 mL/min |
| (therapeutic, non-recirculating) | | |
| Therapeutic Solution Flow Rate | 20 mL/min | 500 mL/min |
| (debridement, recirculating) | | |

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

We claim:

1. A system for treating a burn wound caused by a burning trauma, the system comprising:
a Hydrodynamic Dressing (HD) configured and adapted to enclose the burn wound;
three distinct Therapeutic Fluids (TFs), each respectively formulated to support a designated stage of healing with respect to the burn wound; and
an Automated Fluid Delivery System (AFDS) configured and adapted to deliver each of the three TFs, respectively, to the burn wound enclosed within the HD at a respectively specified time, temperature, pressure, and flow rate according to a burn wound treatment protocol.

2. The system according to claim 1, wherein the HD comprises a wound enclosure that isolates the wound from the nosocomial environment, facilitates wound observation, and facilitates hydrodynamic debridement.

3. The system according to claim 2, wherein the AFDS comprises an automated conditioning and pumping unit that is configured and adapted to deliver each of the three TFs, respectively, to the burn wound enclosed within the HD in sequence, at specified flow rates, pressures, and temperatures.

4. The system according to claim 3, wherein the HD comprises a nozzle configured and adapted to deliver one or more of the three distinct TFs at a measured rate to facilitate the removal of toxins and necrotic byproducts from the burn wound.

5. The system according to claim 4, wherein the HD comprises a distributed array of nozzles, each respective nozzle configured and adapted to deliver one or more of the three distinct TFs at a measured rate to facilitate the removal of toxins and necrotic byproducts from the burn wound.

6. The system according to claim 5, wherein the HD comprises an outlet port configurable to route fluids that pass across the burn wound enclosed within the HD to either a waste receptacle or a recirculating pump.

7. The system according to claim 3, wherein the three distinct TFs comprise:
a first TF formulated to generate an optimal wound healing environment within 0 to 3 days after burning trauma;
a second TF formulated to generate an optimal wound healing environment within 3 to 5 days after burning trauma; and
a third TF formulated to generate an optimal wound healing environment within 6 to 12 days after burning trauma.

8. The system according to claim 7, wherein a combination of the first TF, the second TF, and the third TF is formulated to provide intense hydration conducive to wound healing, to restore normal osmotic pressure in the burned tissue, to chemically and hydrodynamically micro-debride and flush away necrotic byproducts and oxidizing compounds, to remove bacterial biofilm, to maintain a hygienic wound environment, and to inhibit one or more undesirable effects selected from the list consisting of inflammation, edema, and excessive fibrous tissue proliferation.

9. The system according to claim 8, wherein:
the first TF is formulated to:
remove coagulated and dead tissue from the burn wound,
perform hydrodynamic micro-chemical debridement on the burn wound,
increase tissue perfusion to remove pro-inflammatory soluble components within an interstitial fluid formed with an edema on the burn wound,
mitigate bacterial biofilm formation on the burn wound,
purify toxic substances from the burn wound, and
reduce pain from the burn wound;
the second TF is formulated to:
remove flaking from burnt skin layers on the burn wound,
perform hydrodynamic micro-chemical debridement on the burn wound,
restore isotonic condition in the burn wound,
promote a topical anti-edematous and anti-inflammatory action on the burn wound,
mitigate bacterial biofilm growth on the burn wound, and
reduce pain from the burn wound; and
the third TF is formulated to:
modulate the activation and conversion of resident skin fibroblasts into myofibroblasts in response to burn injury,
promote a reduction of hypertrophic scars, keloids, and fibrosis on the burn wound,
promote a more homogeneous wound healing without stains and roughness on the burn wound,
promote a reduction of contractures and loss of fine motor movement on the burn wound,
promote a topical anti-edematous and anti-inflammatory action on the burn wound, and
mitigate bacterial biofilm growth on the burn wound.

10. The system according to claim 9, wherein the burn wound treatment protocol comprises:
administration of the first TF between 12 to 15° C. and within 48 to 72 hours after burning trauma;
administration of the second TF between 15 to 25° C. and within 3 to 5 days after burning trauma; and
administration of the third TF between 25 to 30° C. and within 6 to 9 days after burning trauma.

11. The system according to claim 10, wherein the burn wound treatment protocol comprises:
administering the first TF in conjunction with venous hydration therapy for hydration to replace fluid and electrolyte losses, following recommendations of the American Burn Association, comprising:
administering formal fluid resuscitation at a rate of 2-4 (ml/kg body weight) divided by percent total body surface area (TBSA) burned during the first 24 hours; and
titrating fluid resuscitation to maintain a urine output of approximately 0.5-1.0 ml/kg/hour in an adult patient over 17 years of age or 1.0-1.5 ml/kg/hour in a pediatric patient 17 years of age or younger.

12. A method of treating a burn wound caused by a burning trauma, the method comprising the following steps:
providing a Hydrodynamic Dressing Pouch (HD) configured and adapted to enclose the burn wound;
providing three distinct Therapeutic Fluids (TFs), each respectively formulated to support a designated stage of healing with respect to the burn wound;
providing an Automated Fluid Delivery System (AFDS) comprising a wound enclosure that isolates the wound from the nosocomial environment, facilitates wound observation, and facilitates hydrodynamic debridement; and
delivering each of the three TFs, respectively, to the burn wound enclosed within the HD at a respectively specified time, temperature, pressure, and flow rate according to a burn wound treatment protocol.

13. The method according to claim 12, wherein the step of delivering each of the three TFs, respectively, to the burn wound enclosed within the HD comprises bringing each of the three TFs, respectively and sequentially, to a respective specified pressure and temperature at a respective specified time within an automated conditioning and pumping unit before or during delivering each of the three TFs, respectively, to the burn wound enclosed within the HD.

14. The method according to claim 13, comprising removal of toxins and necrotic byproducts from the burn wound by action of a nozzle on the burn wound enclosed within the HD.

15. The method according to claim 13, wherein the three distinct TFs comprise:
a first TF formulated to generate an optimal wound healing environment within 0 to 3 days after burning trauma;
a second TF formulated to generate an optimal wound healing environment within 3 to 5 days after burning trauma; and
a third TF formulated to generate an optimal wound healing environment within 6 to 9 days after burning trauma.

16. The method according to claim 15, wherein a combination of the first TF, the second TF, and the third TF is formulated to provide intense hydration conducive to wound healing, to restore normal osmotic pressure in the burned tissue, to chemically and hydrodynamically micro-debride and flush away necrotic byproducts and oxidizing compounds, to remove bacterial biofilm, to maintain a hygienic wound environment, and to inhibit one or more undesirable effects selected from the list consisting of inflammation, edema, and excessive fibrous tissue proliferation.

17. The method according to claim 16, wherein:
the first TF is formulated to:
remove coagulated and dead tissue from the burn wound,
perform hydrodynamic micro-chemical debridement on the burn wound,
increase tissue perfusion to remove pro-inflammatory soluble components within an interstitial fluid formed with an edema on the burn wound,
mitigate bacterial biofilm formation on the burn wound,
purify toxic substances from the burn wound, and
reduce pain from the burn wound;
the second TF is formulated to:
remove flaking from burnt skin layers on the burn wound,
perform hydrodynamic micro-chemical debridement on the burn wound,
restore isotonic condition in the burn wound,
promote a topical anti-edematous and anti-inflammatory action on the burn wound,
mitigate bacterial biofilm growth on the burn wound, and
reduce pain from the burn wound; and
the third TF is formulated to:
modulate the activation and conversion of resident skin fibroblasts into myofibroblasts in response to burn injury,
promote a reduction of hypertrophic scars, keloids, and fibrosis on the burn wound,
promote a more homogeneous wound healing without stains and roughness on the burn wound,
promote a reduction of contractures and loss of fine motor movement on the burn wound,
promote a topical anti-edematous and anti-inflammatory action on the burn wound, and
mitigate bacterial biofilm growth on the burn wound.

18. The method according to claim 17, wherein the burn wound treatment protocol comprises:
administration of the first TF between 12 to 15° C. and within 48 to 72 hours after burning trauma;
administration of the second TF between 15 to 25° C. and within 3 to 5 days after burning trauma; and
administration of the third TF between 25 to 30° C. and within 6 to 9 days after burning trauma.

19. The method according to claim 18, wherein the burn wound treatment protocol comprises:
administering the first TF in conjunction with venous hydration therapy for hydration to replace fluid and electrolyte losses, following recommendations of the American Burn Association, comprising:
administering formal fluid resuscitation at a rate of 2-4 (ml/kg body weight) divided by percent total body surface area (TBSA) burned during the first 24 hours; and
titrating fluid resuscitation to maintain a urine output of approximately 0.5-1.0 ml/kg/hour in an adult patient over 17 years of age or 1.0-1.5 ml/kg/hour in a pediatric patient 17 years of age or younger.

20. A system for treating a burn wound caused by a burning trauma, the system comprising:
a Hydrodynamic Dressing Pouch (HD) configured and adapted to enclose the burn wound;
three distinct Therapeutic Fluids (TFs), each respectively formulated to support a designated stage of healing with respect to the burn wound; and
an Automated Fluid Delivery System (AFDS) configured and adapted to deliver each of the three TFs, respectively, to the burn wound enclosed within the HD at a respectively specified time, temperature, pressure, and flow rate according to a burn wound treatment protocol;
wherein the HD comprises a wound enclosure that isolates the wound from the nosocomial environment, facilitates wound observation, and facilitates hydrodynamic debridement;
wherein the AFDS comprises an automated conditioning and pumping unit that is configured and adapted to deliver each of the three TFs, respectively, to the burn wound enclosed within the HD in sequence, at specified flow rates, pressures, and temperatures;
wherein the HD comprises a distributed array of nozzles, each respective nozzle configured and adapted to deliver one or more of the three distinct TFs at a measured rate to facilitate the removal of toxins and necrotic byproducts from the burn wound;
wherein the HD comprises an outlet port configurable to route fluids that pass across the burn wound enclosed within the HD to either a waste receptacle or a recirculating pump;
wherein the three distinct TFs comprise:
a first TF formulated to generate an optimal wound healing environment within 0 to 3 days after burning trauma;
a second TF formulated to generate an optimal wound healing environment within 3 to 5 days after burning trauma; and
a third TF formulated to generate an optimal wound healing environment within 6 to 9 days after burning trauma;
wherein a combination of the first TF, the second TF, and the third TF is formulated to provide intense hydration conducive to wound healing, to restore normal osmotic pressure in the burned tissue, to chemically and hydrodynamically micro-debride and flush away necrotic byproducts and oxidizing compounds, to remove bacterial biofilm, to maintain a hygienic wound environment, and to inhibit one or more undesirable effects selected from the list consisting of inflammation, edema, and excessive fibrous tissue proliferation;
wherein the first TF is formulated to:
remove coagulated and dead tissue from the burn wound,
perform hydrodynamic micro-chemical debridement on the burn wound,
increase tissue perfusion to remove pro-inflammatory soluble components within an interstitial fluid formed with an edema on the burn wound,
mitigate bacterial biofilm formation on the burn wound,
purify toxic substances from the burn wound, and
reduce pain from the burn wound;
wherein the second TF is formulated to:
remove flaking from burnt skin layers on the burn wound,
perform hydrodynamic micro-chemical debridement on the burn wound,
restore isotonic condition in the burn wound,
promote a topical anti-edematous and anti-inflammatory action on the burn wound,
mitigate bacterial biofilm growth on the burn wound, and
reduce pain from the burn wound; and
wherein the third TF is formulated to:
modulate the activation and conversion of resident skin fibroblasts into myofibroblasts in response to burn,
promote a reduction of hypertrophic scars, keloids, and fibrosis on the burn wound,
promote a more homogeneous wound healing without stains and roughness on the burn wound,
promote a reduction of contractures and loss of fine motor movement on the burn wound,
promote a topical anti-edematous and anti-inflammatory action on the burn wound, and
mitigate bacterial biofilm growth on the burn wound;
wherein the burn wound treatment protocol comprises:
administration of the first TF between 12 to 15° C. and within 48 to 72 hours after burning trauma;
administration of the second TF between 15 to 25° C. and within 3 to 5 days after burning trauma; and
administration of the third TF between 25 to 30° C. and within 6 to 9 days after burning trauma; and
wherein the burn wound treatment protocol comprises administering the first TF in conjunction with venous hydration therapy for hydration to replace fluid and electrolyte losses, comprising:
administering formal fluid resuscitation at a rate of 2-4 (ml/kg body weight) divided by percent total body surface area (TBSA) burned during the first 24 hours; and
titrating fluid resuscitation to maintain a urine output of approximately 0.5-1.0 ml/kg/hour in an adult patient over 17 years of age or 1.0-1.5 ml/kg/hour in a pediatric patient 17 years of age or younger.
